(19) United States
(12) Patent Application Publication
(10) Pub. No.: US 2016/0289780 A1
(43) Pub. Date: Oct. 6, 2016

(54) METHODS FOR ENERGY-EFFICIENT HIGH SOLIDS LIQUEFACTION OF BIOMASS

(71) Applicants: THE REGENTS OF THE UNIVERSITY OF CALIFORNIA, Oakland, CA (US); ASPECT AI LTD., Shoham (IL)

(72) Inventors: Maria Cardona, Davis, CA (US); Robert Louis Powell, III, Davis, CA (US); Michael John McCarthy, Davis, CA (US); Tina Jeoh Zicari, Davis, CA (US); Emilio Javier Tozzi, West Chester, OH (US)

(73) Assignees: THE REGENTS OF THE UNIVERSITY OF CALIFORNIA, Oakland, CA (US); ASPECT AI LTD., Shoham (IL)

(21) Appl. No.: 15/090,898

(22) Filed: Apr. 5, 2016

Related U.S. Application Data
(63) Continuation of application No. PCT/US2014/059940, filed on Oct. 9, 2014.
(60) Provisional application No. 61/888,836, filed on Oct. 9, 2013.

(51) Int. Cl. C12Q 3/00; C12P 19/02; C12P 19/14; G01N 11/04; G01N 24/08

(52) U.S. Cl. CPC C12Q 3/00; G01N 11/04; G01N 24/088; G01N 24/085; C12P 19/14; C12P 19/02; C12P 2201/00

(57) ABSTRACT

The present disclosure is generally related a method for the liquefaction of high-solids biomass substrates. Particularly, biomass can be added to a reactor until a pressure drop, measured inline, reaches the maximum system limitations. A commercial enzyme mixture (specific for the particular type of biomass to be processed) may then be added to the biomass, forming a slurry. The pressure may be continuously monitored and when the pressure drop reaches a steady state (which can be determined by little or no change in pressure drop for several minutes), more biomass may then be added until the high pressure limit of the pump system is reached again. The method can be repeated until the desired quantity of biomass is processed.

### CROSS-REFERENCE TO RELATED APPLICATIONS

This application is a 35 U.S.C. §111(a) continuation of PCT international application number PCT/US2014/059940 filed on Oct. 10, 2014, incorporated herein by reference in its entirety, which claims priority to, and the benefit of, U.S. provisional patent application Ser. No. 61/888,836 filed on Oct. 9, 2013, incorporated herein by reference in its entirety. Priority is claimed to each of the foregoing applications.

The above-referenced PCT international application was published as PCT International Publication No. WO 2015/054519 on Apr. 16, 2015, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technical Field

This disclosure pertains generally to the processing of biomass, and more particularly to a controlled fed-batch reaction process for the energy-efficient enzymatic liquefaction of biomass at high solids concentrations.

2. Background Discussion

The enzymatic hydrolysis of cellulosic biomass is a necessary step for the sustainable production of fuels and chemicals. The reaction consists of the enzymatic breakdown of cellulose components of biomass and these reactions are traditionally carried out in slurries containing biomass, cellulase enzyme mixtures, water, and buffer as a means to maintain reaction pH.

The amount of water and buffer in the mixture influences processing costs. High amounts of water result in large energy usages in distillation steps downstream, for instance. Because of this, industrial-level implementation of the aforementioned process is desirable at solids concentrations greater than 15% (w/w) to reduce the excess amount of water present while still keeping the reaction viable. At such high solids concentrations, however, the mixture becomes highly viscous, which results in difficulties mixing, pumping and maintaining appropriate mass and heat transfer in the slurry. It has been shown that appropriate mixing throughout the reaction is crucial for efficient liquefaction and hydrolysis. This means that for higher productivity, the enzymatic hydrolysis of biomass should be carried out at high solids concentrations while still maintaining efficient mixing. Current technologies to handle high solids biomass require specialized equipment (e.g., scraped-surface reactors), and are limited in volume to the available reactor size.

Current methods, in which fed-batch hydrolysis of cellulosic biomass was demonstrated, do not account for energy efficiency when deciding when and how much biomass and enzymes to add to the reactor. For industrial-scale applications, energy efficiency is of high importance. Prior research in fed-batch hydrolysis of biomass has included batch additions based on waiting a fixed amount of time between additions. The traditional approach does not control viscosity, is sub-optimal and is not suited to deal efficiently with variability in biomass or any perturbations that occur during the process, which results in wasted time and energy.

### BRIEF SUMMARY

A method is described that uses rheology as a variable to control the addition of biomass and enzyme to the ongoing liquefaction of biomass. By responding to the changes in viscosity that occur during the liquefaction reaction, the viscosity can be maintained at levels that facilitate mixing and heat transfer. Specifically, biomass substrate and enzymes can be added to a reactor until the measured pressure drop reaches the maximum limitation allowed by the system. The pressure can be continuously monitored and when the measured pressure drop reaches a quasi steady state after a measured decrease in pressure, more biomass can be added to the reactor.

One aspect of the presently disclosed method is the incorporation of non-invasive measurements of energy efficiency. This energy efficiency evaluation allows for the most energy-efficient enzyme loading policy for any given substrate and enzymes. The controlled enzyme feeding scheme results in lower energy requirements and higher solids throughput in a shorter amount of time. By monitoring the progress of liquefaction, higher overall productivity can be achieved.

Additionally, monitoring of pressure drops and energy efficiency can be performed in situ and non-invasively, which overcomes issues that arise with periodic sampling, and can be readily implemented.

Another aspect of the presently disclosed method is that implementation of the process does not require any custom-made equipment, and is only limited in reaction volume by the reactor size.

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a schematic diagram of an embodiment of a flow loop system that can be used to perform embodiments of the presently disclosed method.

FIG. 2A and FIG. 2B are a flow diagram that illustrates the process of MRI rheology in accordance with the described embodiments.

FIG. 3A is an image of a velocity profile obtained at 8 minutes after the initiation of hydrolysis, according to an embodiment of the presently described method.

FIG. 3B is an image of a velocity profile obtained when pressure readings attain a quasi steady state after a decrease in measured pressure, according to an embodiment of the presently described method.

FIG. 4A through FIG. 4G are images of a variety of velocity profiles that correspond to various flow regimes in the described embodiments.

FIG. 5 is a flow diagram of an exemplary method for high solids biomass processing in accordance with the described embodiments.

FIG. 6 is a graph showing the effect of swelling of pretreated wheat straw prior to the addition of enzyme on wall stress over time.

FIG. 7 is a graph showing the effect of pre-soaking the wheat straw overnight on wall stress over time.

FIG. 8A is an initial velocity profile image for wheat straw substrate showing an initial flat profile.

FIG. 8B is a velocity profile image taken shortly after the addition of enzyme.

FIG. 8C is a velocity profile image showing the wheat straw's continuous profile during hydrolysis.

FIG. 9 is a graph that illustrates the evolution of yield stress over time during hydrolysis for sugar beets, Solka Floc short fibers, Solka Floc long fibers and wheat straw substrates.

FIG. 10 is a graph illustrating the evolution of fiber length during hydrolysis over time for the Solka Floc long fibers, wheat straw and Solka Floc short fibers.

FIG. 11 is a graph illustrating the evolution of fiber width during hydrolysis over time for the Solka Floc long fibers, wheat straw and Solka Floc short fibers.

FIG. 12 is a graph illustrating the evolution of total solids over time for the sugar beets, Solka Floc short fibers, Solka Floc long fibers and wheat straw substrates.

FIG. 13 is a graph illustrating the evolution of yield stress over time for the sugar beets, Solka Floc short fibers, Solka Floc long fibers and wheat straw substrates.

FIG. 14 is a graph illustrating the evolution of total solids over time where the biomass and enzyme were added all at once, initially.

FIG. 15 is a graph illustrating the evolution of yield stress over time where the biomass and enzyme were added all at once, initially.

FIG. 16 is a graph illustrating the change in apparent viscosity and extent of hydrolysis over time for the two types of Solka Floc fibers.

FIG. 17 is a graph illustrating the decoupling of liquefaction and saccharification.

FIG. 18 is an image of the velocity profile for the long Solka Floc substrate (c100) at 0 minutes and 120 minutes and the corresponding micrograph images.

FIG. 19 is an image of the velocity profile for the short Solka Floc substrate (200EZ) at 0 minutes and 120 minutes and the corresponding micrograph images.

FIG. 20 is a graph showing the length of the fibers as a function of extent of hydrolysis for both the short and long Solka Floc fibers.

FIG. 21 is a graph showing changes in fiber width as a function of extent of hydrolysis and illustrates that width stays practically the same during hydrolysis.

FIG. 22 is a graph showing the yield stress compared to total solids processed over time when the enzyme is added batchwise.

FIG. 23 is a graph showing the yield stress compared to total solids processed over time when the enzyme is added all at one time initially.

FIG. 24 is a graph illustrating that the batchwise addition of the enzyme results in higher processing yields over a long period of time.

### DETAILED DESCRIPTION

A controlled fed-batch reaction method has been developed for the enzymatic liquefaction of biomass at high solids concentrations. The feeding scheme (i.e., when reactants and enzymes are loaded into the reactor) in this fed-batch process has been developed with lower energy requirements that can be useful in industrial settings.

Cellulosic biomass can be converted to fuels and chemicals via the “biological route,” using enzymes and microorganisms. Of the cellulases that carry out enzymatic hydrolysis of cellulosic biomass, endocellulases have been shown to be the primary enzymes responsible for the rapid reduction in viscosity during hydrolysis, a process which is better known as liquefaction. This rapid reduction in viscosity is ubiquitous to all hydrolysis carried out with cellulase mixtures that contain endocellulases. The majority of commercially available cellulases thus include these enzymes.

After the rapid change in viscosity due to the endo-activity of cellulases on the biomass, the reaction slurry becomes more pumpable and readily mixable. One aspect of the presently described technology is to take advantage of this observed fast drop in viscosity in order to add increasingly higher solids concentrations without running into mixing and pumping issues. High solids concentrations are achieved by adding more biomass in a batchwise manner (without further addition of buffer or acid and base); with each batch added once sufficient liquefaction has taken place in the reactor. This allows for high solids loadings with low viscosity, increasing the production while using less water and energy in the process.

Viscosities of biomass suspensions can be measured using an inline magnetic resonance imaging (MRI) rheometer. Velocity profiles of the slurry flowing in a pipe can be obtained and, in conjunction with pressure drop measurements in the pipe, can be used to obtain rheograms for the slurry in situ. These in situ rheograms can be used to monitor changes in viscosity of biomass as it undergoes liquefaction, and the high speed of the measurements (1-2 minutes per velocity profile image) allows for practically instantaneous viscosity measurements. Therefore, using the fast viscosity measurements as a monitoring tool, the point at which the slurry has undergone sufficient liquefaction can be precisely determined for further additions of biomass and enzyme.

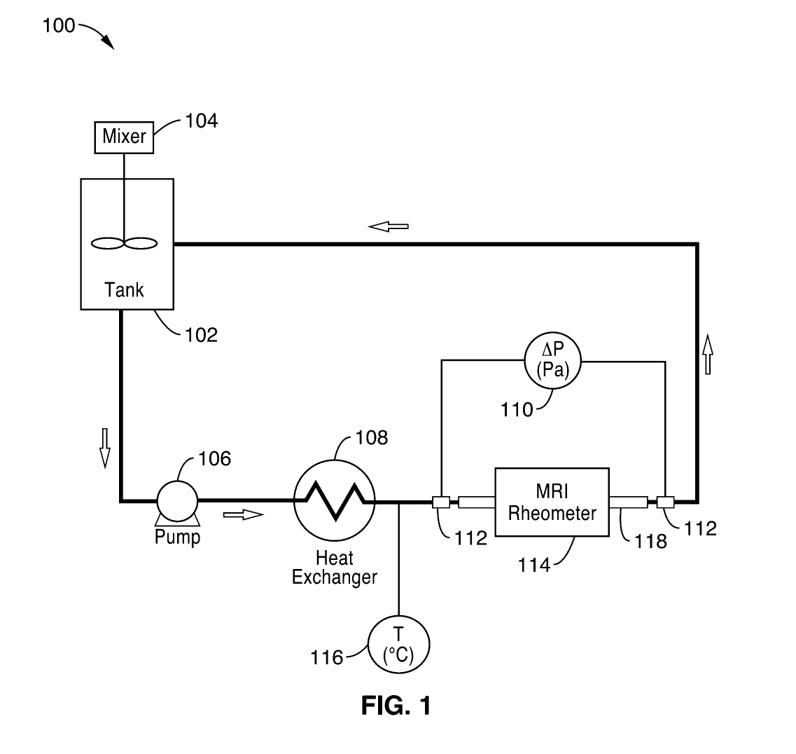

Referring to FIG. 1, in one embodiment, the methods may be carried out in a flow loop system 100. The flow loop may include a tank 102 with an impeller mixer 104, a pump 106, a heat exchanger 108, a pressure transducer 110 with pressure taps 112, an MRI rheometer 114, a temperature monitor (e.g., a thermocouple) 116 and a pipe 118 to transport the material through the flow loop system. Although an MRI rheometer was used in this and the following examples, the process can be carried out by simply using a pressure monitoring system, or with inline measurements of wall stress. Using an MRI rheometer or alternative noninvasive methods (e.g., ultrasound velocimetry, laser Doppler velocimetry) allows for better control and measurement of fluid rheology, but is not necessary for carrying out the presently described process.

A challenge in the measurement of biomass rheology is the presence of large fibers. The fibers tend to settle due to gravity and can be large in size. These characteristics make them unsuitable for measurement using conventional rheometers. Non-invasive rheometers such as MRI rheometers, can handle materials containing large particles.

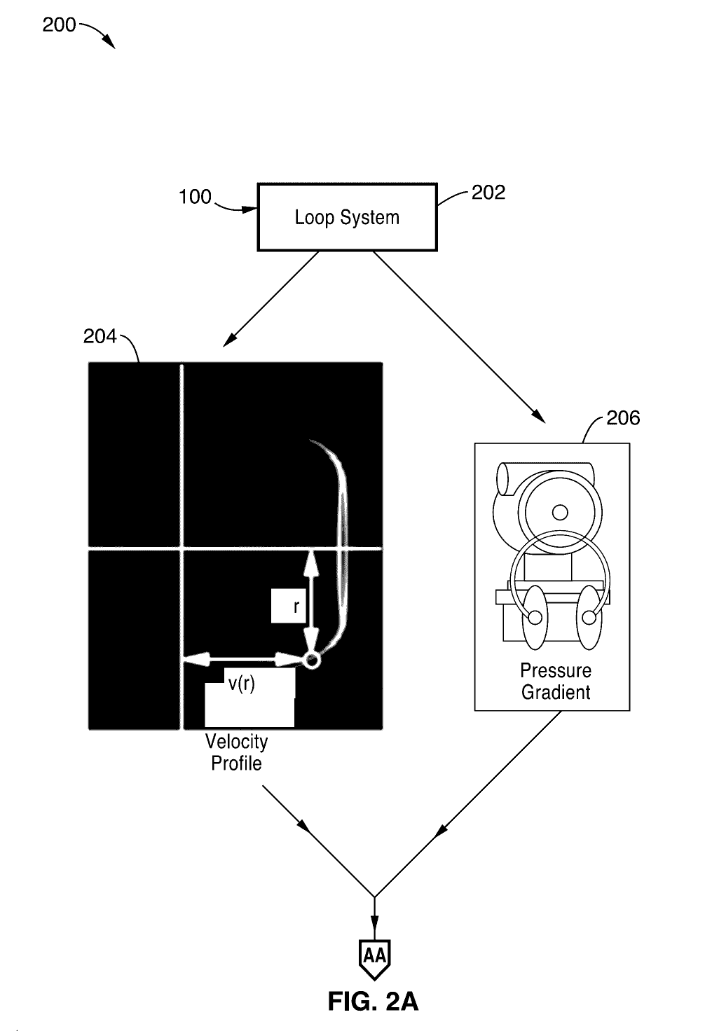

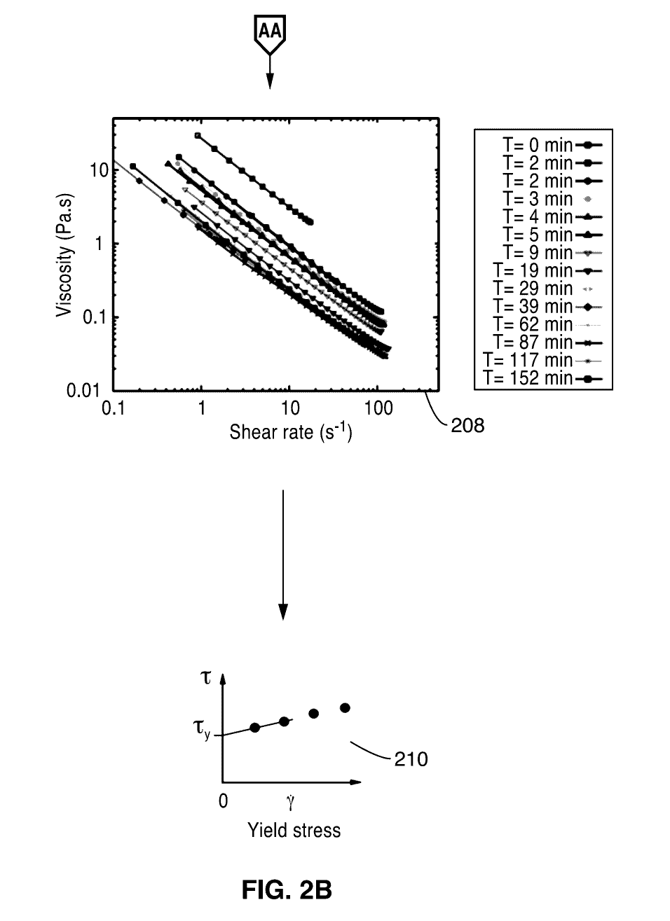

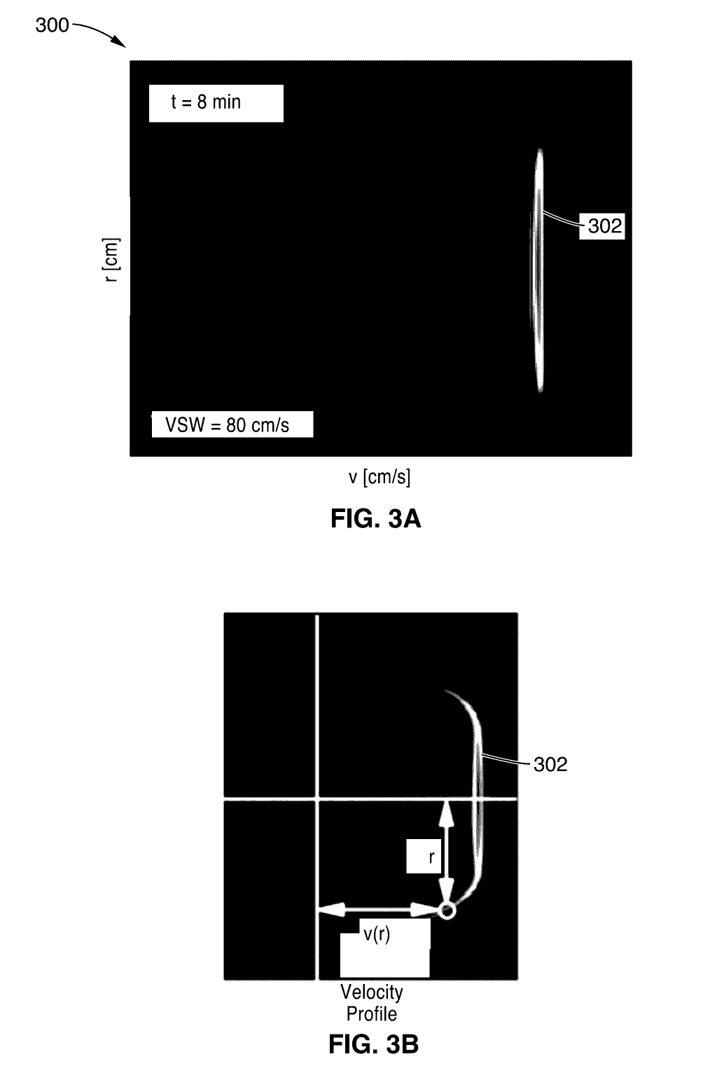

Yield stress is a particularly important property for biomass, since biomass slurries are non-Newtonian in behavior at high solids concentrations. Yield stress is defined as the minimum shear stress that a fluid must experience before it will flow. FIG. 2A and FIG. 2B are a flow diagram 200 illustrating MRI rheology. In this embodiment, the slurry fluid may flow into the magnet in the loop system 202 where it is imaged. Velocity profiles 204 may then be obtained. FIG. 3A and FIG. 3B show velocity profiles 300 obtained at different time points during hydrolysis. FIG. 3A shows the velocity profile at t=8 minutes and FIG. 3B shows the velocity profile at t=120 minutes. The brightest region of the image 302 is the actual velocity profile, which is given as velocity as a function of radial position.

Referring back to FIG. 2A and FIG. 2B, the velocity profiles may then be differentiated and, by incorporating pressure drop measurements taken within the pipe of the flow loop from one point along the pipe to a second point along the pipe 206, a rheogram 208, which reveals the relationship between shear rate and shear stress, may be generated. Yield stress values 210 may also be obtained by extrapolating to 0 shear rate.

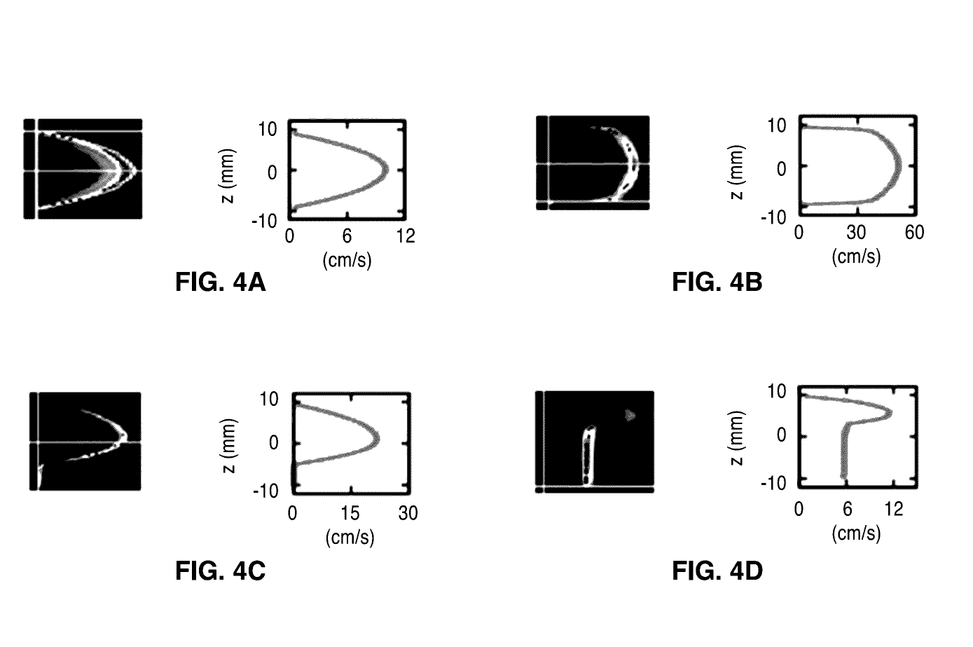

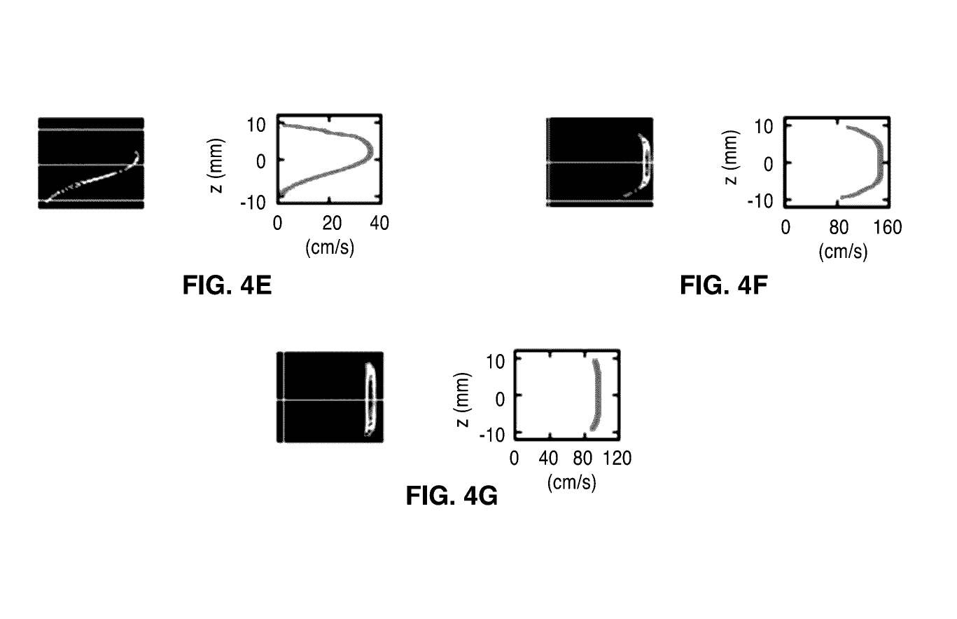

FIG. 4A through FIG. 4G show a variety of velocity profiles that correspond to various flow regimes. FIG. 4B shows turbulent flow, which has a recognizable profile. FIG. 4C, FIG. 4D and FIG. 4E display vertical asymmetry due to particles settling. FIG. 4F and FIG. 4G show typical non-Newtonian behavior of yield stress fluids, characterized by a flat region near the center of the pipe. The MRI rheometer can produce rheological information from profiles such as FIG. 4A, FIG. 4F and FIG. 4G. If flow regimes occur that are not conducive to rheological measurement (such as in FIG. 4C, FIG. 4D and FIG. 4E) or turbulence (FIG. 4B) the presently disclosed method can still detect it.

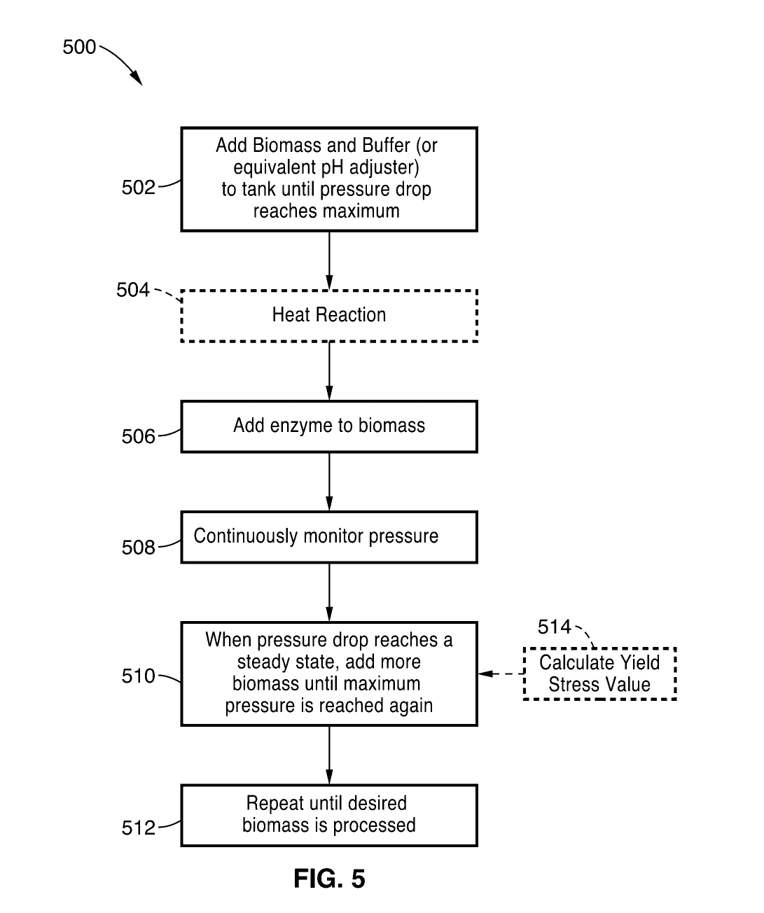

Referring now to FIG. 5, in one embodiment of the presently described process 500, high solids liquefaction can begin by adding biomass and buffer to a tank 502, such as the tank 102 described in FIG. 1, until a pressure drop measured inline (using a pressure transducer, or equivalent instrument) reaches a maximum pressure based on equipment limitations. In this embodiment, the absolute pressure of the entire system is not measured, but rather a pressure difference (i.e., a pressure drop, since fluids flow from a point of high pressure to a point of low pressure) between two points along a section of pipe. This maximum pressure can be determined by constantly monitoring the pressure drop within the pipe (i.e., pressure difference inline) between two points along the pipe. If, for example and without limitation, the components within the flow loop being used can handle approximately 100 kPa, biomass may then be added to the reactor until the measured pressure drop within the pipe between the two points along the pipe reaches 100 kPa. At this point, a commercial enzyme mixture (specific for the type of biomass being processed) may be added to the reactor to perform hydrolysis of the biomass 506.

At any time before the addition of the enzyme, the reaction mixture can be allowed to reach a desired temperature that is optimal for the enzymes being used, such as 50° C. 504. The addition of enzymes to the biomass in the reactor causes an almost instant decrease in the pressure being monitored 508 within the pipe. This pressure may continue to decrease until the measured pressure reaches a plateau, which can be defined as a “quasi steady state” after a decrease in pressure or “low quasi steady state.” The low quasi steady state can be determined by little or no change in the continuously measured pressure drop within the pipe between the two points along the pipe. When the pressure drop reaches a quasi steady state after a decrease in pressure, more biomass may then be added until the high pressure limit of the components within the flow loop is reached again 510.

The term “quasi” steady state can be used in the description of this embodiment because it is not necessary to wait until a “final” steady state, i.e., the state in which the pressure drops to its lowest point without changing, in order to maximize energy efficiency. Therefore, in a preferred embodiment, the first point at which the decrease in measured pressure plateaus (i.e., the low quasi steady state) can be used as a cue to add more biomass. This “low point” or “low quasi steady state” can even be given a set value after the first batch of biomass and enzyme have been added to the reactor. Once the first quasi steady state has been reached, this pressure value may then be used as a cue for adding subsequent batches of biomass.

The steps in boxes 502 through 510 may be repeated until all of the desired solids loadings are added 512. In addition to measuring the pressure, an MRI rheometer can also be used to generate the yield stress value of the slurry 514 (see FIG. 2A and FIG. 2B) which can be used in conjunction with the measured pressure drop to help determine when to add additional enzyme.

The method embodiment described in FIG. 5 may be used to reach high solids concentrations without running into pumping and mixing issues that occur when using standard equipment. Hydrolysis of such high solids concentrations would not be possible if an equivalent amount of biomass is added at one time initially because the high viscosities make it virtually impossible to mix and pump with standard equipment.

The efficiency of the process embodiment described in FIG. 5 may be determined based on the amount of energy dissipated from frictional losses inline. Frictional losses can be defined by the head loss, which is defined as,

$${h}_{f} = {f}_{D} * \frac{L}{D} * \frac{{\overline{V}}^{2}}{2 g} ,$$

better known as the Darcy-Weisbach equation. Cumulative head losses can then be calculated as:

fh_{f}dt,

where h_{f} is the head loss due to friction (in m), f_{D} is the Darcy friction factor (dimensionless and defined as 64/Re for laminar flows, where Re is the Reynolds number), L is the pipe length between pressure taps (in m), D is the pipe diameter (in m), V is the mean velocity (in m/s), g is the gravitational constant (in m/s²), and t is time (in seconds).

Both of these calculations can be used to compare the different enzyme feeding schemes presented herein. The instantaneous energy loss shows the frictional energy losses experienced by the system at any point in time, and the cumulative energy loss shows how much energy was dissipated over a period of time. The former may function as a process control variable, whereas the latter can be used to compare the overall efficiency of the different processes.

Two enzyme feeding schemes were investigated in the examples described herein: (1) all of the enzyme required to hydrolyze the biomass that will be added (total mass from all batches) was added at the beginning of the reaction, and (2) the enzyme was added batchwise, corresponding to the biomass added in each batch. Either scheme of enzyme addition results in the same total amount of enzyme added for the whole process, but results indicated that scheme (1) resulted in higher energy efficiency, as demonstrated by faster batch additions (leading to higher solids concentrations in a shorter amount of time than scheme (2) and lower energy losses from energy dissipation. Scheme (2) lead to slightly higher sugar release but with reduced efficiency in liquefaction (as defined by the rate at which viscosity drops in the reaction slurry). Embodiments of the presently disclosed method can thus include optimization based on desired outcomes of the reaction. In other words, this process separates liquefaction (the reduction in viscosity of the biomass) from hydrolysis (the conversion of cellulose to sugars) depending on the enzyme feeding scheme used.

### EXAMPLE 1

The evolution of yield stress during hydrolysis was examined using four types of biomass: wheat straw, two delignified wood fibers called Solka Floc and sugar beets. The first three are mainly cellulosic. Sugar beets contain substantial amounts of soluble sugars. Table 1 lists the experimental conditions.

For this example, the method embodiment described in FIG. 5 was carried out on a loop system, such as that described in FIG. 1. Specifically, the substrate biomass was loaded into the tank, along with a buffer and the enzyme. Volumes used range from about 5 kg to about 15 kg. The buffer used was a 50 mM sodium citrate buffer at a pH of 5.0. The volume of buffer used is directly scalable with the total solids and equipment volume (e.g., 6.5 L of buffer were added with about 680 g of biomass and 10% (w/w) solids at the beginning of one experiment. The % solids (w/w) refers to (the weight of biomass/total weight of material in the reactor)*100%.

There are also further distinctions, such as soluble versus insoluble solids. In this embodiment, total solids (soluble+insoluble) were evaluated. A practical definition of total solids is based on a test where all the water (and trace amount of volatiles) is removed by evaporation, where Mi is the initial mass of the sample before evaporation of the water, Mf is the final mass of the sample after evaporation of the water and the percent total solids is equal to (Mf/Mi)*100 (i.e., % solids=100-moisture content. The moisture content of the fibers was measured using a Mettler-Toledo model HR83 Halogen Moisture Analyzer (Mettler-Toledo International, Columbus, Ohio).

In this embodiment, the tank contained an impeller (i.e., a rotor used to increase or decrease the pressure flow of a fluid), which allowed the mixture of biomass, buffer and enzyme to be well mixed throughout the process. Samples for use in measuring soluble sugar concentrations or fiber length were taken from the tank.

Contents of the tank were then pumped by a positive displacement pump into the loop at a rate of approximately 9 kg/min, which allowed the mixture to recirculate in approximately 2 minutes. It should be noted, however, that the pumping rate may be varied according to specific process parameters.

Once the enzyme has begun to process the biomass, the mixture becomes a slurry. The slurry was then pumped through a heat exchanger. The heat exchanger used in this embodiment consisted of a stainless steel coil through which the slurry flowed, wherein the coil was submerged in a heated water bath. The coil heat exchanger allowed for the reaction temperature to be maintained from about 45° C. to about 55° C. without temperature fluctuations resulting from changes in room temperature. It should be noted, however, that some thermostable enzymes can work at temperatures from about 60° C. to about 65° C.

The slurry was then pumped into the magnet via a pipe with an inner diameter of about 2 cm, where the slurry was imaged. Although the temperature within the pipe was monitored, this is not necessary. However, measuring the pressure drops in the pipe is necessary for determining the shear stress the fluid is experiencing. The fluid was then recirculated back to the tank.

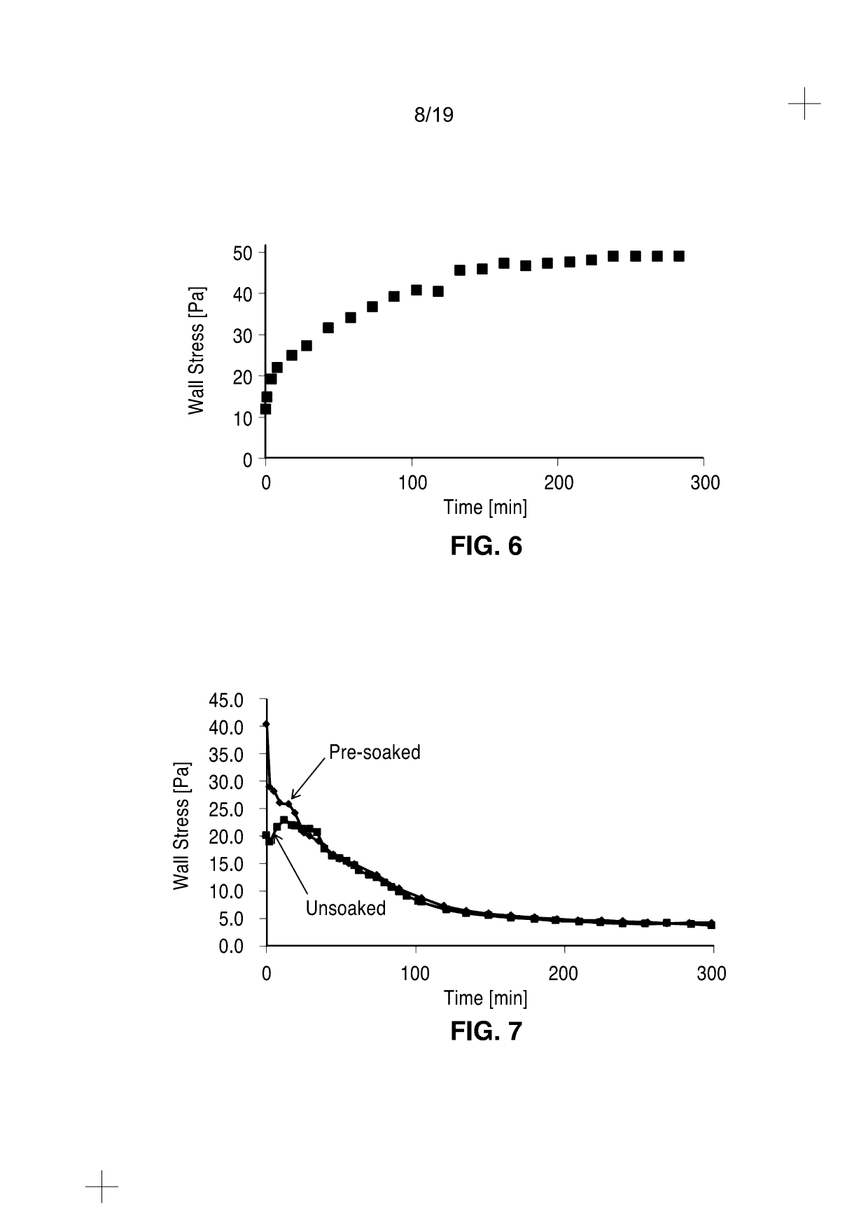

Referring now to FIG. 6, for the wheat straw substrate, it was observed that the rheology changed before adding enzymes due to swelling. The swelling was caused by the interaction of the fibers with water. It was found that pre-soaking the wheat fibers prior to adding enzyme made a difference on initial rheology. The process was performed with and without soaking the wheat fibers overnight. As shown in FIG. 7, the soaking only made a difference on the first few minutes of the process.

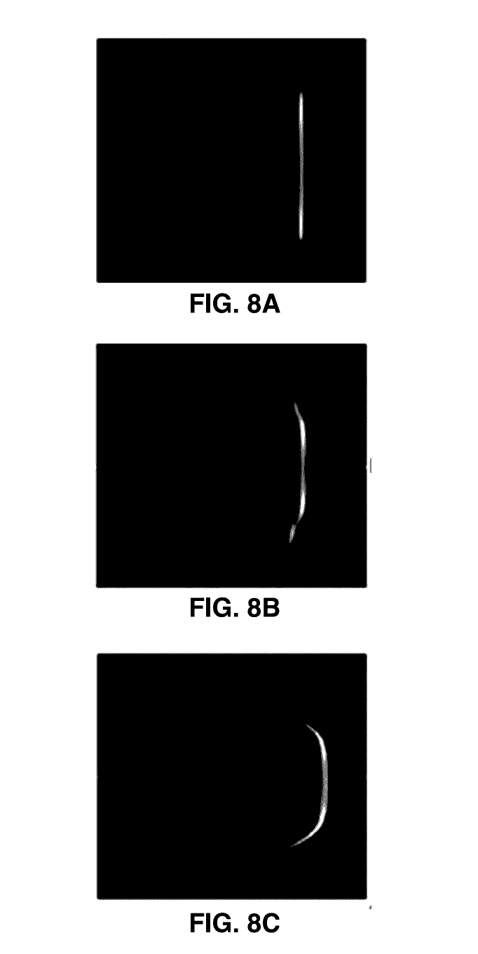

Turning now to FIG. 8A through FIG. 8C, an annular flow velocity profile was observed briefly for the wheat straw. This complex velocity profile was observed shortly after adding the enzyme. This phenomenon was brief and did not interfere with measurements taken during hydrolysis. FIG. 8A shows the initial flat profile, FIG. 8B shows the brief annular profile shortly after enzyme addition and FIG. 8C shows the continuous profile during hydrolysis.

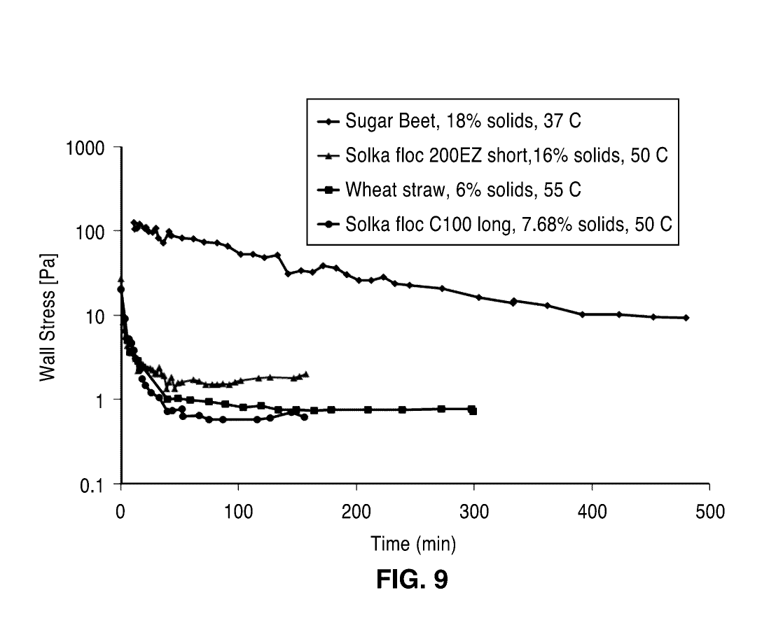

FIG. 9 shows the evolution of yield stress over time during hydrolysis for each substrate. These runs were performed in batch mode by initially adding all the biomass at once.

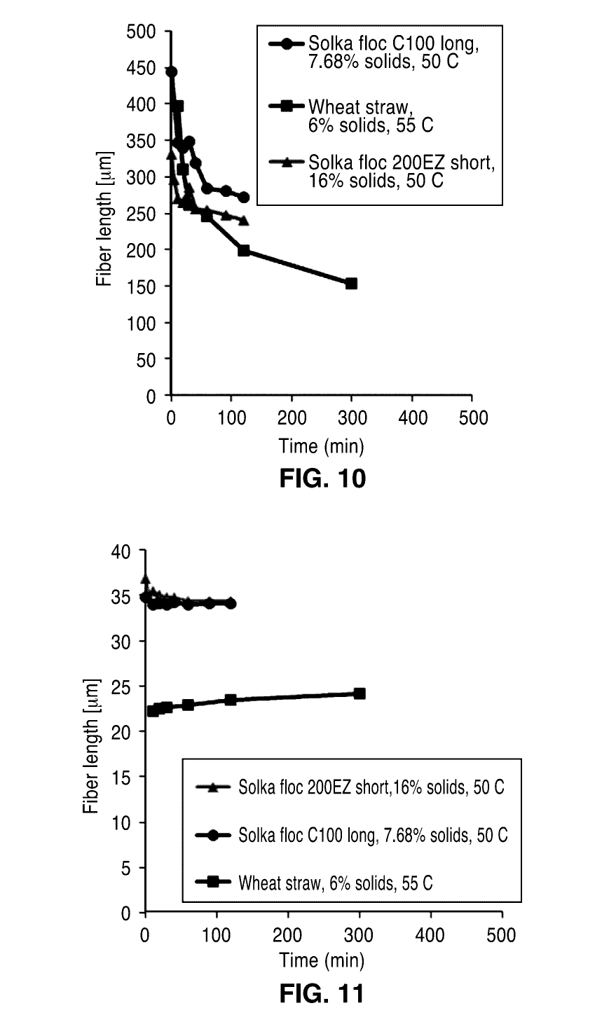

FIG. 10 and FIG. 11 show the evolution of fiber length and width respectively, during hydrolysis over time for each cellulosic substrate. Fiber length changed more rapidly than width, suggesting a mechanism wherein fibers break at weak points before reducing their diameter significantly.

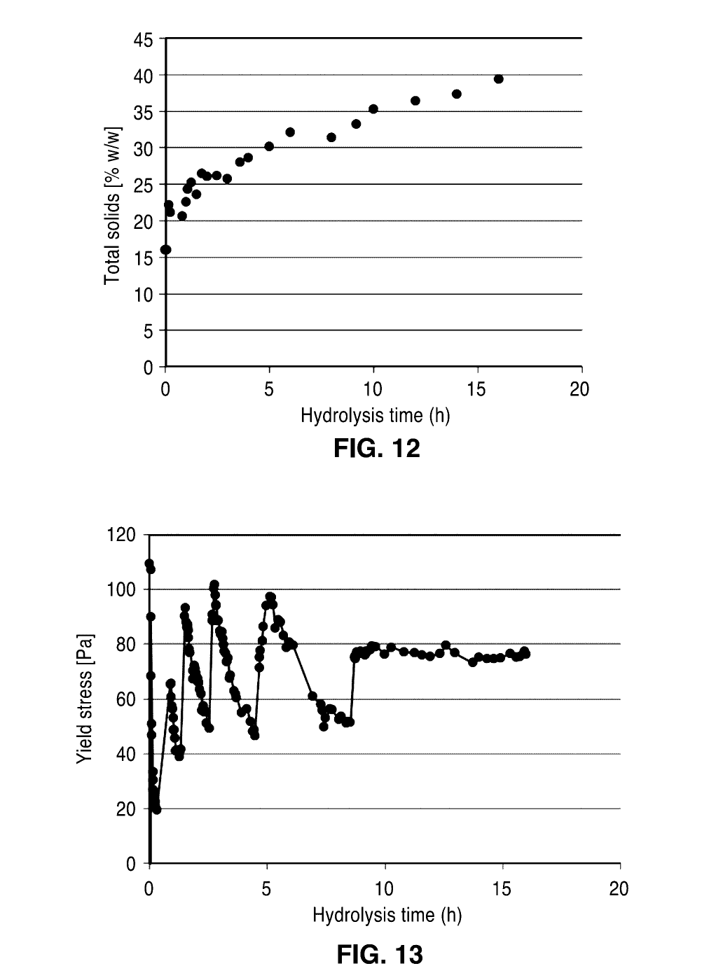

FIG. 12 and FIG. 13 show the evolution of total solids and yield stress over time, respectively, for one of the Solka Floc fibers, where the biomass and enzyme were added batchwise. This run was operated in fed batch mode and the concentration of solids reached 40%. Thus, the process qualified as a “high-solids” process. The experimental conditions were slightly different than those shown in Table 1. Enzyme loading was performed at 5 FPU/g. The enzyme, CTEC2 was added batchwise and the biomass used was Solka Floc 200EZ (short fibers). Referring to FIG. 13, the maximum pressure measured inline according to equipment limitations was 64 kPa. The low quasi steady state was set at 12 kPa after the first batch of biomass was added. Biomass was therefore added until the measured pressure inline reached 64 kPa and allowed to hydrolyze until the measured pressure inline dropped to 12 kPa, at which point more biomass was added again until 64 kPa was reached. The process was repeated until all of the biomass batches had been loaded.

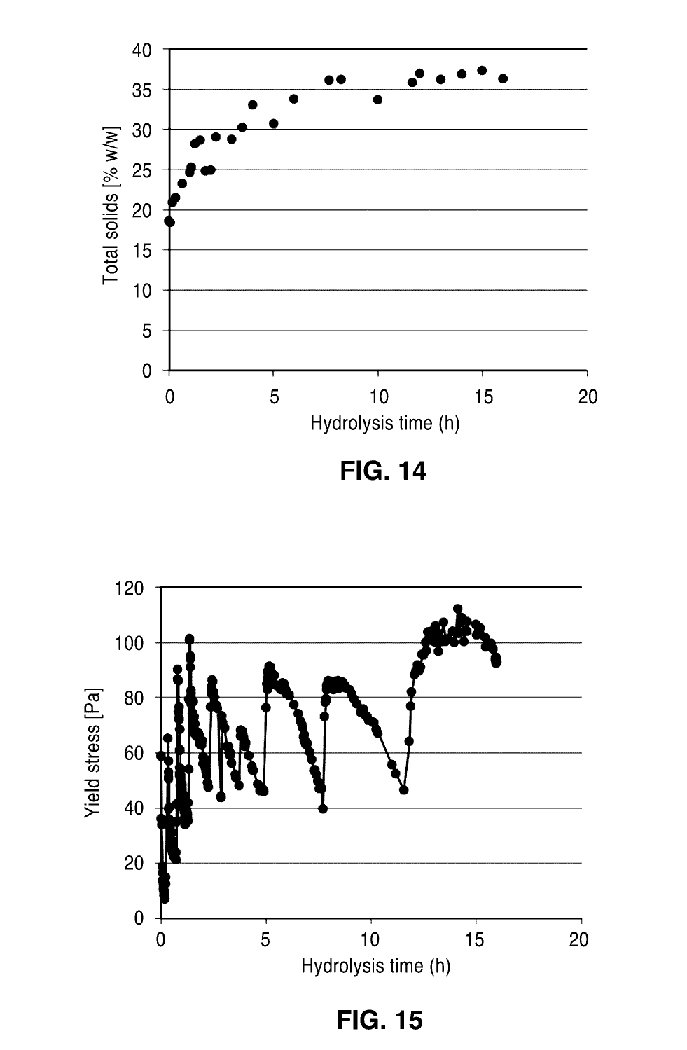

FIG. 14 and FIG. 15 show the evolution of total solids and yield stress over time, respectively, for one of the Solka Floc fibers, where the biomass and enzyme were added all at once. The run was operated in fed batch mode and the concentration of solids reached was 35%. Thus, the process qualified as a “high-solids” process. The experimental conditions were slightly different than those shown in Table 1. Enzyme loading was performed at 5 FPU/g. The enzyme, CTEC2, was added all at once and the biomass used was Solka Floc 200EZ (short fibers).

In this example, yield stress was measured with sub-minute resolution during hydrolysis of various biomass substrates. The decrease in yield stress that was observed over time correlates with fiber shortening. Consequently, it is feasible to measure yield stress rapidly for biomasses, using rheology as a control scheme.

### EXAMPLE 2

In this example, the rheology of 2 types of biomass was examined: a purified form of fibrous cellulose (Solka Floc) and a lignocellulosic substrate (wheat straw). Table 2 shows a summary of the experimental conditions that were used. The method embodiment described in FIG. 5 was also used in this example.

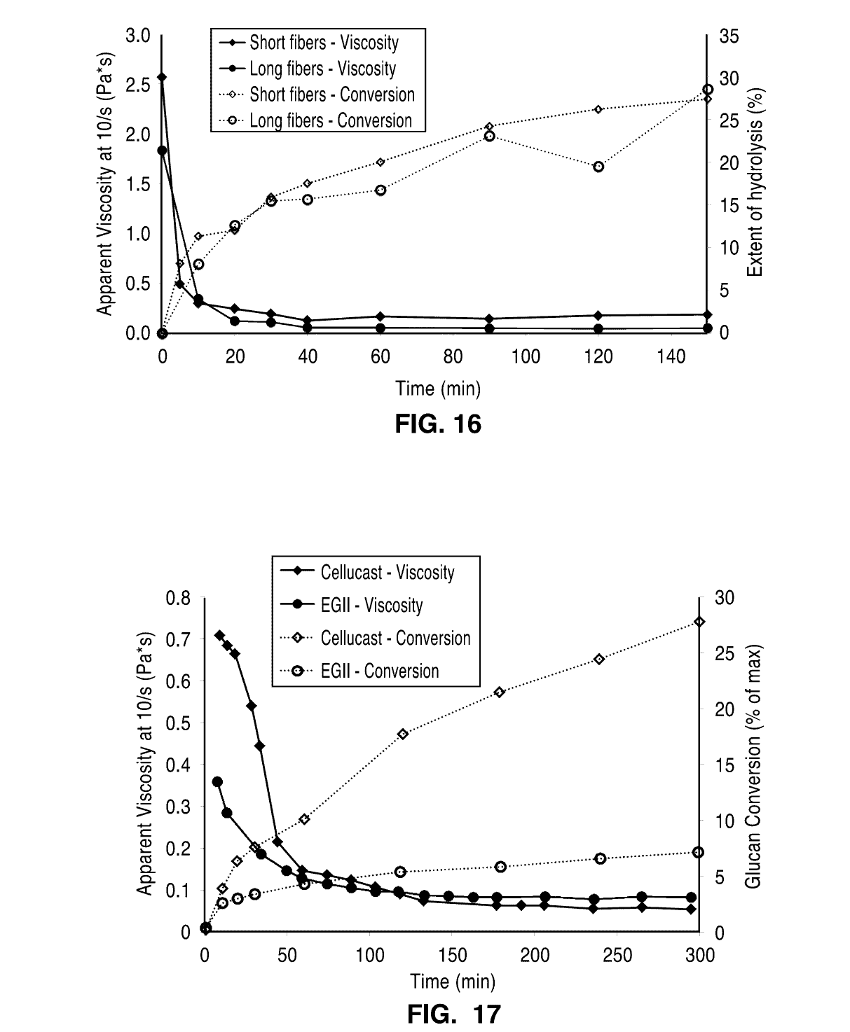

FIG. 16 shows the change in apparent viscosity and extent of hydrolysis over time for the two types of Solka Floc. The graph illustrates that most of the liquefaction took place in the first 20 minutes. Although these substrates are at very different solids loadings (16% for the short fibers and 8% for the long fibers), the substrates behave very similarly during both liquefaction and saccharification. One possibility for this is that both of the substrates started with similar crowding numbers.

FIG. 17 shows how liquefaction and saccharification can be decoupled. This graph illustrates the comparison of liquefaction of wheat straw using a commercial enzyme cocktail (celluclast) versus purified endoglucanase. It is known that the endoglucanase enzyme has the biggest impact on the viscosity of the slurry. As the enzyme cleaves the fibers, the shorter fibers decrease the viscosity of the slurry. FIG. 17 shows a graph of the viscosity and glucan conversion of wheat straw over a period of 5 hours. The graph illustrates that the wheat straw liquefied faster with the purified endoglucanase than it did with the celluclast. However, when comparing glucan conversion, the purified endoglucanase yielded very little sugars when compared to celluclast, as would be expected due to the lack of enzymes like cellobiohydrolases in the pure endoglucanase.

In addition, most of the change in viscosity occurs within the first hour of hydrolysis, at which point very little conversion has occurred. In other words, liquefaction is practically complete before a significant amount of sugars are released. Consequently, the rate of liquefaction does not equal the rate of saccharification, as each is due to different enzyme acting mechanisms. This is of high importance for future kinetic modeling where changes in viscosity must be taken into account, as the changes in viscosity cannot be directly correlated with product generation without properly accounting for enzyme action.

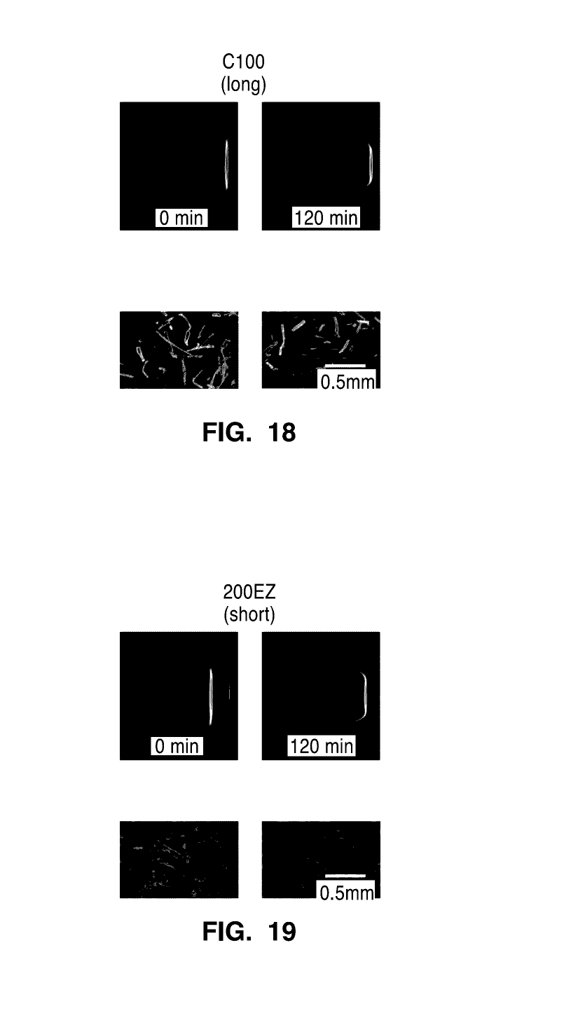

FIG. 18 and FIG. 19 are images showing how the length and width of fibers change during hydrolysis. FIG. 18 shows the velocity profile for the long Solka Floc substrate (C100) at 0 minutes and 120 minutes and the corresponding micrograph images below. FIG. 19 shows the velocity profile for the short Solka Floc substrate (200EZ) at 0 minutes and 120 minutes and the corresponding micrograph images below. The velocity profiles for both substrates are very similar at time 0 and 2 hours.

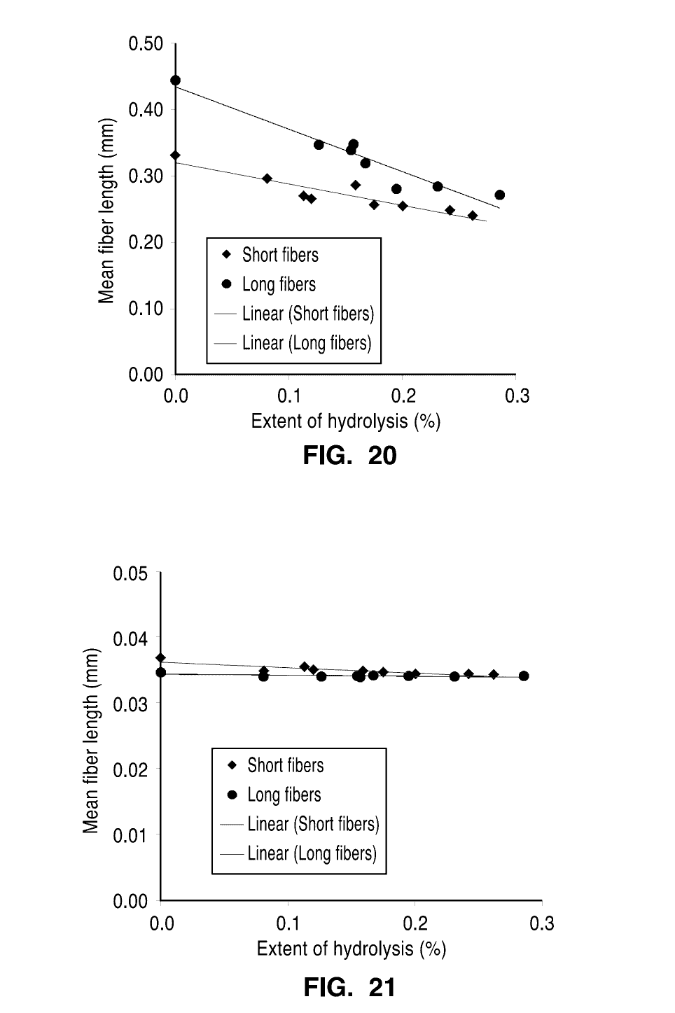

FIG. 20 is a graph showing the length of the fibers as a function of extent of hydrolysis for both the short and long Solka Floc fibers. This graph illustrates the linear relationship between fiber length and fiber conversion. In other words, fiber length is a function of the starting average length and the extent of hydrolysis.

On the other hand, FIG. 21 shows changes in fiber width as a function of extent of hydrolysis and illustrates that width stays practically the same during hydrolysis. As expected, the dimension that changes the most is length. If these results are combined to look at the changes in aspect ratio, which is the ratio of length to width, as a function of conversion, a linear relationship arises wherein the aspect ratio is a function of the starting aspect ratio of each fiber and conversion. From FIG. 20 and FIG. 21:

l=l₀*(1−b*X);

N=2/3φaᵣ²; and

aᵣ=a_{r,0}*(1−b*X)

where φ is the volume fraction, aᵣ is the aspect ratio, X is the conversion and l is the length.

Using these results (liquefaction happens quickly, liquefaction can be seen in average fiber length changes, and liquefaction and saccharification are decoupled), a process was designed which allows for high solids conversion with standard equipment. Specifically, a fed-batch process utilizing the fast drop in viscosity and equipment limitations was developed that is process-based instead of time-based. Using measured high and low wall stress to determine when to add more biomass ensures maximum efficiency for the conversion of biomass while the slurry remains a pumpable consistency.

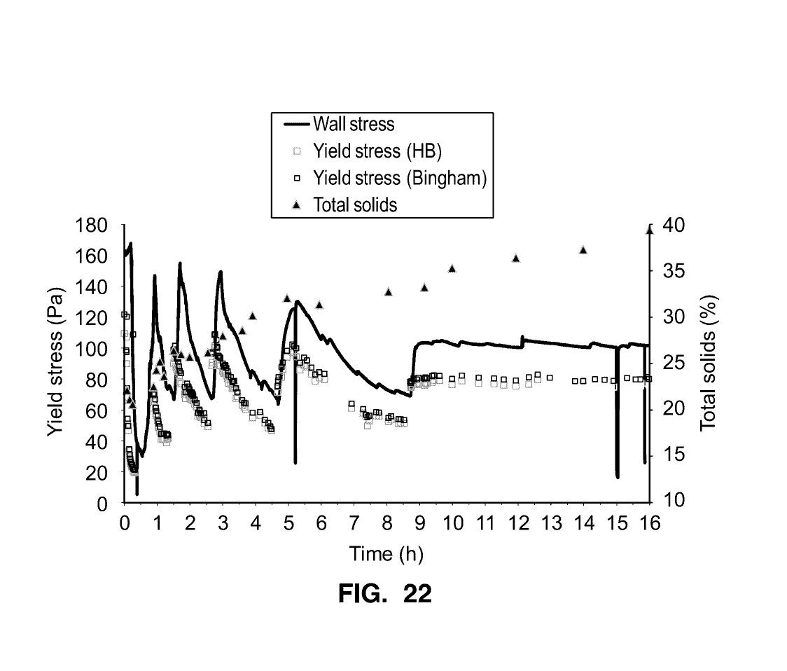

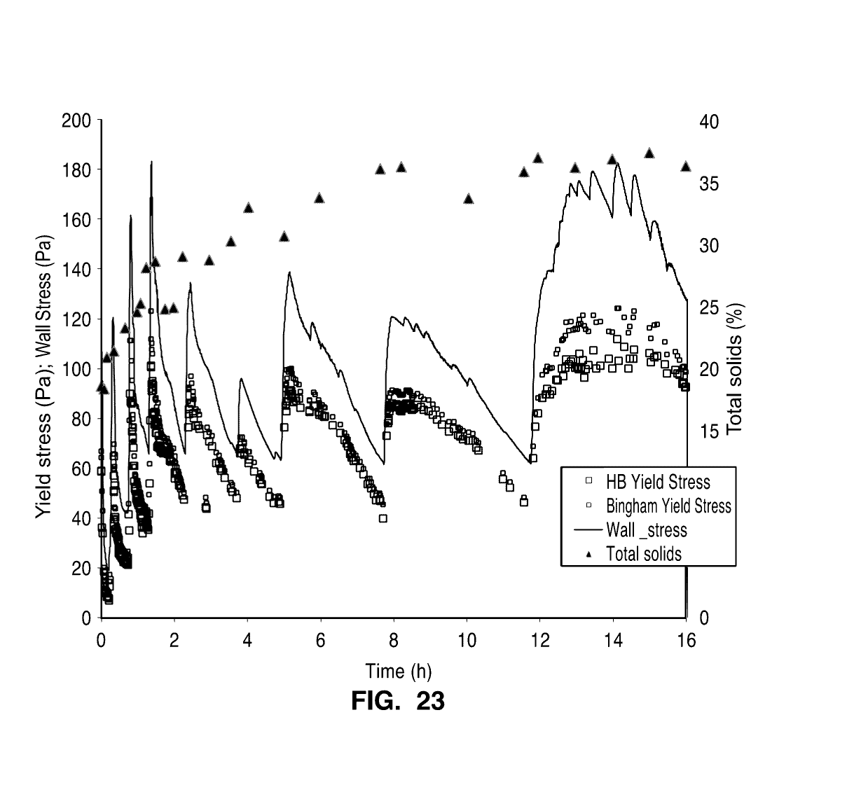

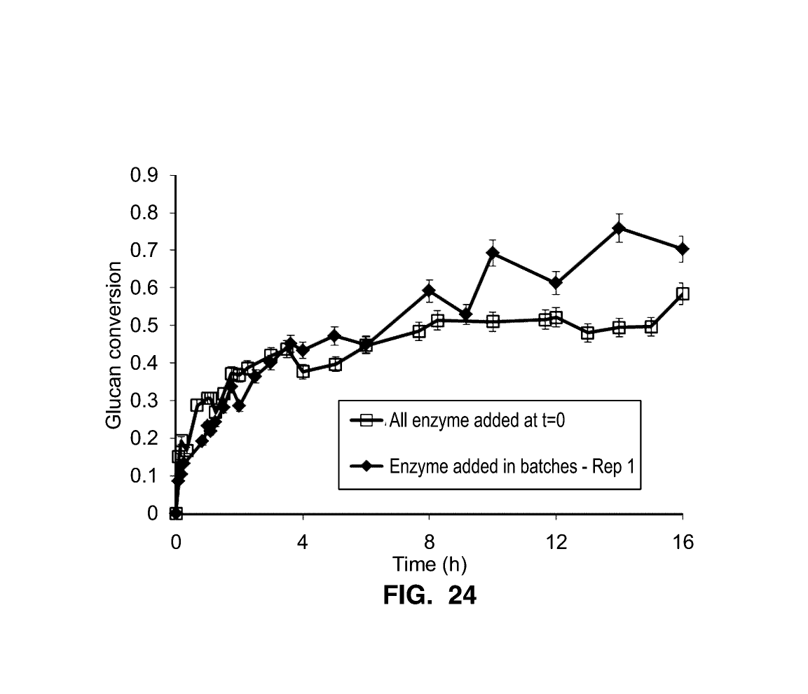

FIG. 22 is a graph showing the yield stress compared to total solids processed over time when the enzyme is added batchwise. On the other hand, FIG. 23 is a graph showing the yield stress compared to total solids processed over time when the enzyme is added all at one time initially. Adding the enzyme batchwise allows for the addition of more batches of biomass, reaching higher solids concentrations faster. Furthermore, FIG. 24 shows that the batchwise addition of the enzyme results in higher processing yields over a long period of time.

Lower solids loadings can indeed be performed to process biomass. However, this results in additional incurred processing costs. The presently disclosed method can achieve high solids concentrations while avoiding mixing and pumping difficulties that arise with highly viscous liquids without specialized equipment.

From the description herein, it will be appreciated that the present disclosure encompasses multiple embodiments which include, but are not limited to, the following:

1. A method of processing biomass, the method comprising: mixing a first batch of biomass and an enzyme to form a first slurry with a first viscosity; adding a second batch of biomass to the first slurry to form a second slurry with a second viscosity; and adding enzyme to the second slurry to form a third slurry with about the first viscosity.

2. A method of processing biomass, the method comprising: (a) providing a reactor with components configured in a flow loop, the components comprising: (i) a tank with a mixer; (ii) a pump to move material through the flow loop; and (iii) a pressure transducer to measure a pressure drop within a pipe that connects the components to form the flow loop, wherein the flowing material flows within the pipe; (b) loading a first batch of biomass into the tank, monitoring a measured pressure drop within the pipe between a first point along the pipe and a second point along the pipe as the biomass is loaded into the tank, and ceasing the loading when the measured pressure drop within the pipe between the first point along the pipe and the second point along the pipe is at a maximum based on the flow loop's limitations; (c) adding an enzyme to the tank and using the mixing the enzyme with the biomass to form a first slurry; (d) monitoring the measured pressure drop within the pipe between the first point along the pipe and the second point along the pipe as the slurry is formed; (e) when the measured pressure drop within the pipe between the first point along the pipe and the second point along the pipe reaches a quasi steady state after a decrease in the measured pressure drop within the pipe between the first point along the pipe and the second point along the pipe, adding an additional batch of biomass into the tank until the measured pressure drop within the pipe between the first point along the pipe and the second point along the pipe is at the maximum based on the flow loop's limitations; (f) adding additional enzyme to the tank and mixing the enzyme with the biomass to form a second slurry; (g) wherein pH of each slurry is maintained at an approximately constant value by adding a buffer or by adding at least one of an acid and a base to each slurry; and (h) repeating steps (e) through (g) until a desired quantity of biomass is processed.

3. The method of any preceding embodiment, wherein the reactor further comprises: an MRI rheometer configured within the flow loop for obtaining velocity profiles of the slurries; wherein the method further comprises: obtaining velocity profiles of the slurries while in the pipe using the MRI rheometer; constructing a rheogram from the velocity profiles and from the pressure drop measured within the pipe between the first point along the pipe and the second point along the pipe as the slurries are formed; calculating a yield stress value from the rheogram; and using the calculated yield stress value to determine when to add additional enzyme to the tank.

4. The method of any preceding embodiment, wherein the velocity profiles of the slurries are obtained every 1 to 2 minutes.

5. The method of any preceding embodiment, wherein the first and second slurries have a solids concentration of at least 15% w/w.

6. The method of any preceding embodiment: wherein the reactor further comprises: a heat exchanger configured within the flow loop for heating the slurries; and wherein the method further comprises: heating the slurries to a temperature of between about 45° C. and 65° C.

7. The method of any preceding embodiment, wherein the biomass comprises cellulosic material and wherein the enzyme comprises endo-cellulase.

8. A method of processing biomass, the method comprising: (a) providing a reactor with components configured in a flow loop, the components comprising: (i) a tank with a mixer; (ii) a pump to move material through the flow loop; and (iii) a pressure transducer to measure a pressure drop within a pipe that connects the components to form the flow loop and wherein the material flows within the pipe; (iv) a MRI rheometer; (b) loading a first batch of biomass and buffer into the tank, monitoring measured pressure drop within the pipe between the first point along the pipe and the second point along the pipe as the biomass is loaded into the tank, and ceasing the loading when the measured pressure drop within the pipe between the first point along the pipe and the second point along the pipe is at a maximum based on the flow loop's limitations; (c) adding an enzyme to the tank and mixing the enzyme with the biomass to form a first slurry; (d) monitoring the measured pressure drop within the pipe between the first point along the pipe and the second point along the pipe as the slurry is formed; (e) when the measured pressure drop within the pipe between the first point along the pipe and the second point along the pipe reaches a quasi steady state after a decrease in the measured pressure drop within the pipe between the first point along the pipe and the second point along the pipe, adding an additional batch of biomass into the tank until the measured pressure drop within the pipe between the first point along the pipe and the second point along the pipe is at the maximum based on the flow system's limitations; (f) obtaining velocity profiles of the slurries while in the pipe using the MRI rheometer; (g) constructing a rheogram from the velocity profiles and from the pressure drop measured within the pipe between the first point along the pipe and the second point along the pipe as the slurries are formed; (h) calculating a yield stress value from the rheogram; (i) using the calculated yield stress value to determine when to add additional enzyme to the tank; (j) adding additional enzyme to the tank and mixing the enzyme with the biomass to form a second slurry; (k) wherein pH of each slurry is maintained at a constant value by adding a buffer or by adding at least one of a base and an acid to each slurry; and (I) repeating steps (d) through (k) until a desired quantity of biomass is processed.

9. The method of any preceding embodiment, wherein the velocity profiles of the slurries are obtained every 1 to 2 minutes.

10. The method of any preceding embodiment, wherein the first and second slurries have a solids concentration of at least 15% w/w.

11. The method of any preceding embodiment: wherein the reactor further comprises: a heat exchanger configured within the flow loop for heating the slurries; and wherein the method further comprises: heating the slurries to a temperature of between about 45° C. and 65° C.

12. The method of any preceding embodiment, wherein the biomass comprises cellulosic material and wherein the enzyme comprises endo-cellulase.

13. A method of processing biomass in a reactor with components configured in a flow loop, the components comprising: a tank with a mixer, a pump to move material through the flow loop and a pressure transducer to measure a pressure drop within a pipe between a first point along the pipe and a second point along the pipe, the pipe connecting the components to form the flow loop, wherein the material flows within the pipe, the method comprising: (a) loading a first batch of biomass into the tank, monitoring a measured pressure drop within the pipe between the first point along the pipe and the second point along the pipe as the biomass is loaded into the tank, and ceasing the loading when the measured pressure drop within the pipe between the first point along the pipe and the second point along the pipe is at a maximum based on the flow loop's limitations; (b) adding an enzyme to the tank and mixing the enzyme with the biomass to form a first slurry; (c) monitoring the measured pressure drop within the pipe between the first point along the pipe and the second point along the pipe as the slurry is formed; (d) when the measured pressure drop between the first point along the pipe and the second point along the pipe reaches a quasi steady state after a decrease in the measured pressure drop within the pipe between the first point along the pipe and the second point along the pipe, adding an additional batch of biomass into the tank until the measured pressure within the pipe between the first point along the pipe and the second point along the pipe is at the maximum based on the flow system's limitations; (e) adding additional enzyme to the tank and mixing the enzyme with the biomass to form a second slurry; (f) wherein pH of each slurry is maintained at an approximately constant value by adding a buffer or by adding at least one of an acid and a base to each slurry; and (g) repeating steps (e) through (g) until a desired quantity of biomass is processed.

14. The method of any preceding embodiment, wherein the method further comprises: obtaining velocity profiles of the slurries while in the pipe using a MRI rheometer configured within the flow loop; constructing a rheogram from the velocity profiles and from the pressure drop measured within the pipe between the first point along the pipe and the second point along the pipe as the slurries are formed; calculating a yield stress value from the rheogram; and using the calculated yield stress value to determine when to add additional enzyme to the tank.

15. The method of any preceding embodiment, wherein the velocity profiles of the slurries are obtained every 1 to 2 minutes.

16. The method of any preceding embodiment, wherein the first and second slurries have a solids concentration of at least 15% w/w.

17. The method of any preceding embodiment: wherein the reactor further comprises: a heat exchanger configured within the flow loop for heating the slurries; and wherein the method further comprises: heating the slurries to a temperature of between about 45° C. and 65° C.

18. The method of any preceding embodiment, wherein the biomass comprises cellulosic material and wherein the enzyme comprises endo-cellulase.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Experimental Conditions for the Evaluation of
the Evolution of Yield Stress During Hydrolysis

| Biomass | Wheat Straw | Solka Floc | Solka Floc | Sugar Beets |
|---|---|---|---|---|
| Type | Steam | C100, long | 200EZ, short | Ground, |
| | Pretreated | fibers | fibers | Autoclaved |
| | (184° C., | | | |
| | 12 min) | | | |
| Solids % | 6% | 7.68% | 16% | 18% |
| Source | Inbicon | International | International | UC Davis |
| | (Denmark) | Fiber Corp. | Fiber Corp. | |
| Temperature | 55 | 50 | 50 | 37 |
| (° C.) | | | | |
| Flow Rate | 9 kg/min | 9 kg/min | 9 kg/min | 9 kg/min |
| Enzyme | 10 mg/g | 15 FPU/g | 15 FPU/g | 4 FPU/g TS, |
| Loading | dry matter | cell. | cell. | 30 XU/g TS, |
| | | | | 20 PGU/g TS |
| Enzyme | Purified | Accelerase | Accelerase | CTEC2 + |
| Type | Endo- | 1500 | 1500 | HTEC2 |
| | glucanase | | | NS22119 |
| | from | | | Pectinase |
| | T. Reesei | | | |

TABLE 2
Experimental Overview of Liquefaction

| Biomass | Solka Floc | | Wheat Straw |
|---|---|---|---|
| Type | C100, long fibers | 200EZ, short fibers | Steam Pretreated |
| Solids % | 7.68% | 16% | 6% |
| Temperature | 50 | 50 | 55 |
| (° C.) | | | |
| Flow Rate | 9 kg/min | 9 kg/min | 9 kg/min |
| Enzyme | 15 FPU/g | 15 FPU/g | 10 mg/g |
| Loading | cell | cell. | dry matter |
| Enzyme | Accelerase, | Accelerase, | Various |
| Type | 1500 | 1500 | (Celluclast, EGII |
| | | | from T. |
| | | | aurantiacus) |

What is claimed is:

1. A method of processing biomass, the method comprising:
mixing a first batch of biomass and an enzyme to form a first slurry with a first viscosity;
adding a second batch of biomass to the first slurry to form a second slurry with a second viscosity; and
adding enzyme to the second slurry to form a third slurry with about the first viscosity.

2. A method of processing biomass, the method comprising:
(a) providing a reactor with components configured in a flow loop, the components comprising:
a tank with a mixer;
(ii) a pump to move material through the flow loop; and
(iii) a pressure transducer to measure a pressure drop within a pipe that connects the components to form the flow loop, wherein the flowing material flows within the pipe;
(b) loading a first batch of biomass into the tank, monitoring a measured pressure drop within the pipe between a first point along the pipe and a second point along the pipe as the biomass is loaded into the tank, and ceasing the loading when the measured pressure drop within the pipe between the first point along the pipe and the second point along the pipe is at a maximum based on the flow loop's limitations;
(c) adding an enzyme to the tank and using the mixing the enzyme with the biomass to form a first slurry;
(d) monitoring the measured pressure drop within the pipe between the first point along the pipe and the second point along the pipe as the slurry is formed;
(e) when the measured pressure drop within the pipe between the first point along the pipe and the second point along the pipe reaches a quasi steady state after a decrease in the measured pressure drop within the pipe between the first point along the pipe and the second point along the pipe, adding an additional batch of biomass into the tank until the measured pressure drop within the pipe between the first point along the pipe and the second point along the pipe is at the maximum based on the flow loop's limitations;
(f) adding additional enzyme to the tank and mixing the enzyme with the biomass to form a second slurry;
(g) wherein pH of each slurry is maintained at an approximately constant value by adding a buffer or by adding at least one of an acid and a base to each slurry; and
(h) repeating steps (e) through (g) until a desired quantity of biomass is processed.

3. The method of claim 2, wherein the reactor further comprises:
an MRI rheometer configured within the flow loop for obtaining velocity profiles of the slurries;
wherein the method further comprises:
obtaining velocity profiles of the slurries while in the pipe using the MRI rheometer;
constructing a rheogram from the velocity profiles and from the pressure drop measured within the pipe between the first point along the pipe and the second point along the pipe as the slurries are formed;
calculating a yield stress value from the rheogram; and
using the calculated yield stress value to determine when to add additional enzyme to the tank.

4. The method of claim 3, wherein the velocity profiles of the slurries are obtained every 1 to 2 minutes.

5. The method of claim 2, wherein the first and second slurries have a solids concentration of at least 15% w/w.

6. The method of claim 2:
wherein the reactor further comprises a heat exchanger configured within the flow loop for heating the slurries; and
wherein the method further comprises heating the slurries to a temperature of between about 45° C. and 65° C.

7. The method of claim 2, wherein the biomass comprises cellulosic material and wherein the enzyme comprises endo-cellulase.

8. A method of processing biomass, the method comprising:
(a) providing a reactor with components configured in a flow loop, the components comprising:
a tank with a mixer;
(ii) a pump to move material through the flow loop; and
(iii) a pressure transducer to measure a pressure drop within a pipe that connects the components to form the flow loop and wherein the material flows within the pipe;
(iv) a MRI rheometer;
(b) loading a first batch of biomass and buffer into the tank, monitoring a measured pressure drop within the pipe between the first point along the pipe and the second point along the pipe as the biomass is loaded into the tank, and ceasing the loading when the measured pressure drop within the pipe between the first point along the pipe and the second point along the pipe is at a maximum based on the flow loop's limitations;
(c) adding an enzyme to the tank and mixing the enzyme with the biomass to form a first slurry;
(d) monitoring the measured pressure drop within the pipe between the first point along the pipe and the second point along the pipe as the slurry is formed;
(e) when the measured pressure drop within the pipe between the first point along the pipe and the second point along the pipe reaches a quasi steady state after a decrease in the measured pressure drop within the pipe between the first point along the pipe and the second point along the pipe, adding an additional batch of biomass into the tank until the measured pressure drop within the pipe between the first point along the pipe and the second point along the pipe is at the maximum based on the flow system's limitations;
(f) obtaining velocity profiles of the slurries while in the pipe using the MRI rheometer;
(g) constructing a rheogram from the velocity profiles and from the pressure drop measured within the pipe between the first point along the pipe and the second point along the pipe as the slurries are formed;
(h) calculating a yield stress value from the rheogram;
using the calculated yield stress value to determine when to add additional enzyme to the tank;
(j) adding additional enzyme to the tank and mixing the enzyme with the biomass to form a second slurry;
(k) wherein pH of each slurry is maintained at a constant value by adding a buffer or by adding at least one of a base and an acid to each slurry; and
(I) repeating steps (d) through (k) until a desired quantity of biomass is processed.

9. The method of claim 8, wherein the velocity profiles of the slurries are obtained every 1 to 2 minutes.

10. The method of claim 8, wherein the first and second slurries have a solids concentration of at least 15% w/w.

11. The method of claim 8:
wherein the reactor further comprises a heat exchanger configured within the flow loop for heating the slurries; and
wherein the method further comprises heating the slurries to a temperature of between about 45° C. and 65° C.

12. The method of claim 8, wherein the biomass comprises cellulosic material and wherein the enzyme comprises endo-cellulase.

13. A method of processing biomass in a reactor with components configured in a flow loop, the components comprising: a tank with a mixer, a pump to move material through the flow loop and a pressure transducer to measure a pressure drop within a pipe between a first point along the pipe and a second point along the pipe, the pipe connecting the components to form the flow loop, wherein the material flows within the pipe, the method comprising:
(a) loading a first batch of biomass into the tank, monitoring a measured pressure drop within the pipe between the first point along the pipe and the second point along the pipe as the biomass is loaded into the tank, and ceasing the loading when the measured pressure drop within the pipe between the first point along the pipe and the second point along the pipe is at a maximum based on the flow loop's limitations;
(b) adding an enzyme to the tank and mixing the enzyme with the biomass to form a first slurry;
(c) monitoring the measured pressure drop within the pipe between the first point along the pipe and the second point along the pipe as the slurry is formed;
(d) when the measured pressure drop between the first point along the pipe and the second point along the pipe reaches a quasi steady state after a decrease in the measured pressure drop within the pipe between the first point along the pipe and the second point along the pipe, adding an additional batch of biomass into the tank until the measured pressure within the pipe between the first point along the pipe and the second point along the pipe is at the maximum based on the flow system's limitations;
(e) adding additional enzyme to the tank and mixing the enzyme with the biomass to form a second slurry;
(f) wherein pH of each slurry is maintained at an approximately constant value by adding a buffer or by adding at least one of an acid and a base to each slurry; and
(g) repeating steps (e) through (g) until a desired quantity of biomass is processed.

14. The method of claim 13, wherein the method further comprises:
obtaining velocity profiles of the slurries while in the pipe using a MRI rheometer configured within the flow loop;
constructing a rheogram from the velocity profiles and from the pressure drop measured within the pipe between the first point along the pipe and the second point along the pipe as the slurries are formed;
calculating a yield stress value from the rheogram; and
using the calculated yield stress value to determine when to add additional enzyme to the tank.

15. The method of claim 14, wherein the velocity profiles of the slurries are obtained every 1 to 2 minutes.

16. The method of claim 13, wherein the first and second slurries have a solids concentration of at least 15% w/w.

17. The method of claim 13:
wherein the reactor further comprises a heat exchanger configured within the flow loop for heating the slurries; and
wherein the method further comprises heating the slurries to a temperature of between about 45° C. and 65° C.

18. The method of claim 13, wherein the biomass comprises cellulosic material and wherein the enzyme comprises endo-cellulase.
